(19) United States
(12) Patent Application Publication
(10) Pub. No.: US 2003/0087293 A1
(43) Pub. Date: May 8, 2003

(54) Nucleic acid isolation method and apparatus for performing same

(75) Inventor: Pall Gestsson, Reykjavik (IS)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: deCODE genetics ehf., Reykjavik (IS)

(21) Appl. No.: 10/268,598

(22) Filed: Oct. 10, 2002

Related U.S. Application Data
(60) Provisional application No. 60/335,531, filed on Oct. 23, 2001.

(51) Int. Cl. C12Q 1/68; C12M 1/34

(52) U.S. Cl. 435/6; 435/287.2

(57) ABSTRACT

Methods for purifying nucleic acids from biological fluids are disclosed as well as an apparatus for preforming the methods. The methods and apparatus circumvent traditional centrifugation steps using a plurality of vacuum steps to separate and isolate nucleic acids. The vacuum processing and apparatus allow the direct collection of bound nucleic acids from to collection vessels avoiding further manipulation.

### RELATED APPLICATION

This application claims the benefit of U.S. Provisional Application No. 60/335,531, filed Oct. 23, 2001. The entire teachings of the above application are incorporated herein by reference.

### BACKGROUND OF THE INVENTION

Methods of isolating nucleic acids from biological materials (e.g., whole blood) typically comprise lysis of biological material by a detergent in the presence of protein degrading enzymes, followed by several extractions with organic solvents, e,g., phenol and/or chloroform, ethanol precipitation and dialysis of the nucleic acids. More recently, nucleic acid purification procedures have exploited the affinity nucleic acids have for solid support materials, such as glass, in the presence of a chaotropic reagent. These known methods of, e.g., isolating (double-stranded) DNA from clinical material are very laborious and time-consuming. The relatively large number of steps and physical handling required to purify nucleic acid from such starting materials increase the risk of transmission of nucleic acids from sample to sample in the simultaneous processing of several clinical samples. When nucleic acids are isolated for the subsequent detection of the presence a particular nucleic acid of, e.g., a pathogen (such as a virus or a bacterium) by means of a nucleic acid amplification method there is an increased risk of such a transmission of nucleic acid between different samples which can causes false positive results. Inaccurate results present a serious drawback. Furthermore, many steps are involved in this process, including multiple centrifugation steps and transfers of the material. This repeated manipulation increases the risk of loss of material and potential cross contamination between sample vessels.

Therefore, the need exists for purifying nucleic acids amenable to high throughput screening and automation that reduces the physical manipulation and handling of the samples and avoids loss of sample and cross contamination.

### SUMMARY OF THE INVENTION

The invention pertains to a method and apparatus utilizing vacuum processing and a nucleic acid binding device for purifying nucleic acids directly into collection vessels without the use of centrifugation.

In particular embodiments, the method of the invention relates to the purification of nucleic acids, such as DNA, RNA, genomic DNA or viral DNA, from biological fluids, particularly whole blood. In another embodiment, the invention relates to the purification of nucleic acids from biological fluids of sample volumes of about 200 μl to about 10 mL.

One aspect of the invention relates to an apparatus for purifying nucleic acids from a biological fluid, comprising a base, an intermediate base and a plurality of receiving vacuum manifolds which are interchangeable depending upon the step performed in the nucleic acid isolation procedure. The elements of the apparatus can be made from milled or molded plastic. The base has a plurality of spaced apart discrete upright cylindrical chambers which vertically intersect the base for receiving a nucleic acid binding device. Each chamber of the base has a top opening and a bottom opening for receiving the nucleic acid binding device held within the chambers with an elastomeric band, the band can be placed around the binding device or located within the chamber. An intermediate base is included and has an equal number of chambers corresponding to the base, each with a top opening and downwardly tapered sides to a bottom opening of sufficient diameter to allow vacuum processing to each chamber while avoiding a detrimental pressure drop in any chamber. As such, the intermediate base regulates the vacuum in each chamber.

A receiving manifold (also referred to herein as the “extraction manifold”) is assembled with the base and the intermediate base in a sandwich configuration for receiving wash reagents and filtrate used in the extraction of nucleic acids. The extraction manifold and intermediate base are used during the nucleic acid extraction steps. For elution and collection of the nucleic acids, the extraction manifold and intermediate base are replaced with a second receiving manifold (also referred to herein as the “collection manifold”). The collection manifold has a plurality of chambers for receiving collection vessels corresponding in position to the base. This apparatus allows the elution of the nucleic acids from the binding device directly into collection vessels. The collection manifold has a channel from the chambers and leading to the vacuum source which is of sufficient diameter to allow vacuum processing to each chamber while avoiding a detrimental pressure drop in any chamber. Each of the interchangeable manifolds (nucleic acid collection manifold and extraction manifold) has an inlet for connection to a vacuum source to maintain vacuum when connected to the base.

The invention also relates to a method for purifying nucleic acids from biological fluids utilizing vacuum processing using an apparatus in an extraction mode and in a collection mode, as described herein. The method includes mixing the biological fluids containing nucleic acids with a protease and lysis buffer to form a solution; incubating the solution, and liberating (e.g., releasing) the nucleic acids into solution; adding the solution to the nucleic acid binding device which is in a chamber of the base in a sandwich configuration with the intermediate base and first receiving manifold, so that the nucleic acids bind to a sorbent in the nucleic acid binding device. The bound nucleic acids on the sorbent are then washed with buffer to remove any contaminants. The wash is collected in the extraction manifold using vacuum conditions. The intermediate base and extraction manifold are then replaced with the collection manifold containing collection vessels forming the collection apparatus. This collection apparatus assembly allows the elution of the nucleic acid from the sorbent directly into a collection vessel under vacuum. The methods of the invention are carried out with vacuum processing without the use of centrifugation.

In certain embodiments, the methods of the invention relate to the purification of nucleic acids accomplished by automated vacuum processing.

### BRIEF DESCRIPTION OF THE DRAWINGS

FIG. 1a is a plan view of a base member for receiving and supporting nucleic acid binding devices.

FIG. 1b is a cross section of the base member taken on the line I-I on FIG. 1a.

FIG. 1c is a side elevation of the nucleic acid binding device.

FIG. 2a is a perspective view of an intermediate base member.

FIG. 2b is a cross section of the intermediate base member taken on the line II-II on FIG. 2a.

FIG. 3a is a perspective view of the receiving extraction manifold.

FIG. 3b is an end view of the receiving extraction manifold.

FIG. 4a is the extraction apparatus, an assembly of the base member, the intermediate base, and the receiving extraction manifold for performing the binding and washing steps.

FIG. 4b is an end view of the assembly of FIG. 4a with a nucleic acid binding device shown in phantom.

FIG. 5a is a plan view of a collection embodiment of the receiving manifold.

FIG. 5b is a cross section of the collection manifold taken on the line V-V of FIG. 5a.

FIG. 5c is a collection vessel for use with the second receiving manifold.

FIG. 6a is a perspective of the collection apparatus, a member and collection manifold assembled for elution and direct collection of nucleic acids into collection vessels.

FIG. 6b is an end view of the base member assembled with the collection manifold, with a binding device shown in phantom.

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

### DETAILED DESCRIPTION OF THE INVENTION

A description of preferred embodiments of the invention follows.

The present invention relates to a method of nucleic acid purification using vacuum processing for direct collection of nucleic acids without the use of centrifugation. Nucleic acid, as described herein, is intended to encompass, DNA, RNA, genomic and viral DNA.

The methods and apparatus of the invention utilize vacuum manifold technology for the enhanced ease of manipulation and direct recovery of nucleic acids. “Manifold” as the term is used herein, refers to an instrument having several outlets or apertures through which a liquid or gas is distributed. The apparatus and method further minimize the number of sample transfers and eliminates centrifugation steps, such as those in traditional nucleic acid purification protocols. Additionally, the methods and apparatus of the invention allow simultaneous processing of multiple samples with safe handling of potentially infectious samples. The processing of large sample volumes can be accomplished with minimal handling.

In one aspect, the invention pertains to a method for isolating nucleic acids. Accordingly, the method comprises: (i) contacting a sample containing nucleic acids with a nucleic acid binding device under vacuum conditions and appropriate buffer conditions to bind the nucleic acids to sorbent located in the device; (ii) washing the device with nucleic acids bound to the sorbent under vacuum conditions to remove any unbound material; and (iii) eluting the nucleic acids from the sorbent on the device using an appropriate buffer to disrupt the binding and allow the nucleic acids to filter through the sorbent of the device directly into collection vessels under vacuum conditions.

The sample containing the nucleic acid can be a biological sample. A biological sample containing nucleic acids includes, but is not limited to, whole blood, plasma, body fluids (e.g., synovial, cerebrospinal, saliva, milk, urine, feces, semen or the like), buffy coat, lymphocytes, cultured cells, tissues such as liver, brain, lung, heart, kidney or spleen. The biological sample can additionally contain whole virus. For example, the whole virus can be selected from the group consisting of hepatitis C virus, hepatitis A virus, hepatitis G virus, human immunodeficiency virus, human T-cell leukemia virus I, human T-cell leukemia virus II, and human lymphotropic virus. Whole virus can be disrupted by lysing according to the method to release the viral RNA desired to be purified. The sample should be prepared for use with the methods of the invention by first lysing the cells to first liberate or release nucleic acids. See Exemplification.

Conventional protocols for obtaining DNA or RNA from cells are well known in the art and are described in, for example, Chapter 2 (DNA) and Chapter 4 (RNA) of F. Ausubel et al., eds., Current Protocols in Molecular Biology, Wiley-Interscience, New York (1993), incorporated herein by reference in its entirety. For DNA, these protocols generally entail gently lysing the cells with solubilization of the DNA and enzymatically or chemically substantially freeing the DNA from contaminating substances such as proteins, RNA and other substances (i.e., reducing the concentrations of these contaminants in the same solution as the DNA to a level that is low enough that the molecular biological procedures of interest can be carried out). For isolation of RNA, the lysis and solubilization procedures must include measures for inhibition of ribonucleases and contaminants to be separated from the RNA including DNA.

In another aspect of the invention, the invention pertains to an apparatus for carrying out the nucleic acid isolation methods described herein. The apparatus comprises a plurality of elements which are used in an interchangeable relationship (i.e., extraction mode and collection mode, utilizing the extraction manifold and subsequently the collection manifold, respectively) to each other and under vacuum conditions such that centrifugation steps common in nucleic acid isolation are eliminated. An extraction apparatus and a collection apparatus are described.

The vacuum processing assembly has a base, with a plurality of chambers for receiving sample or containers therefor (hereinafter “nucleic acid binding device”). The nucleic acid binding device, as described herein, can contain a thin nucleic acid sorbent disk between an upper funnel or top and a lower base. The nucleic acid sorbent disk can be made from any nucleic acid binding material such as silica, glass particles, PVDF, silica gel, diatomaceous earth, glass, aluminum oxides, titanium oxides, zirconium oxides, and hydroxyapatite, ion exchange resins or other material readily available in the art for reversible binding of nucleic acids. A particularly preferred nucleic acid binding device is an QIAamp Maxi column available from Qiagen Inc. (Valencia, Calif.) or other suitable devices or chromatography columns commercially available for nucleic acid purification. An elastomeric band is placed on the outside of the device or located in each chamber of the base for holding the device in the chambers of the base and providing an air tight seal. An example of such a band is an oil seal joint radial manufactured by Chicago Rawhide Inc., Elgin, Ill.

In another embodiment, the binding device can contain two filters positioned sequentially. The first filter is made of an appropriate material, e.g., PVDF, to filter out solid biological debris (e.g., cell debris or cells) and the second filter is a sorbent which binds nucleic acids. It is advantageous to utilize a two filter system with samples that contain cell debris and material that can clog the pores or the nucleic acid sorbent and therefore reduce the potential binding and quantity of nucleic acids recovered.

The nucleic acid binding devices are placed individually in each of the chambers of the base. The number of chambers can be designed to carry out a single separation or multiple separation, such as 12, or in alternate embodiments can be 24, 96, or for use with high-density format plates 384, 864 and 1536. If the chambers are not being utilized for a purification, stoppers (e.g., binding devices, or rubber stoppers) can be positioned such that the vacuum pressure needed to carry out the process in all chambers is maintained. The chambers are vertical chambers and can be arranged in rows and columns in which a nucleic acid binding device is placed within each chamber of the base.

A sample of biological fluid containing nucleic acids is transferred to the binding device under conditions suitable for binding of the nucleic acids to the sorbent contained within the device. The bound nucleic acids are sequentially treated with liquid reagents and washes typically involved in the purification of the nucleic acids. Vacuum pressure is applied to collect the wash buffers. A typical wash buffer used for nucleic acid binding via ionic charge is Tris-HCl at pH 5 with 200 nM sodium chloride and 5 mM EDTA. The wash removes contaminants that bind less tightly than nucleic acids.

The binding device, as described herein, provides the filtering of a solid portion of the sample, such as cell debris (if any) to remain in the top portion of the device, selective absorption of nucleic acids from the sample onto a sorbent located within nucleic acid binding device, and allows the liquid portion of the sample to filter through the sorbent.

After appropriate washing steps, the nucleic acids can be liberated from the sorbent by disrupting the binding under suitable conditions and allowing the nucleic acids to pass through the sorbent and drawn into collection vessels using appropriate vacuum conditions, rather then centrifugation. The apparatus uses standard DNA collection vessels (e.g., 2 ml storage tubes and cryovials). The collection manifold can be adapted to receive a variety of vials used in the art. The bound nucleic acid can be eluted from the sorbent with a minimal amount of an appropriate solvent under conditions which disrupt the affinity the nucleic acid has to the sorbent. Reagents and solvents used to liberate the nucleic acid from the sorbent are well known to those of skill in the art and include but are not limited to Tris-HCl-ethylene diaminetetraacetic acid (TE) buffer or distilled sterile water.

Larger sample volumes can be processed using the methods and apparatus as described herein. Starting sample volumes of between about 200 μl to about 10 mL, can be processed using a 12 chamber configuration, smaller size samples can be processed using other chamber size configurations (e.g., 24, 96 or high throughput arrangements such as 384).

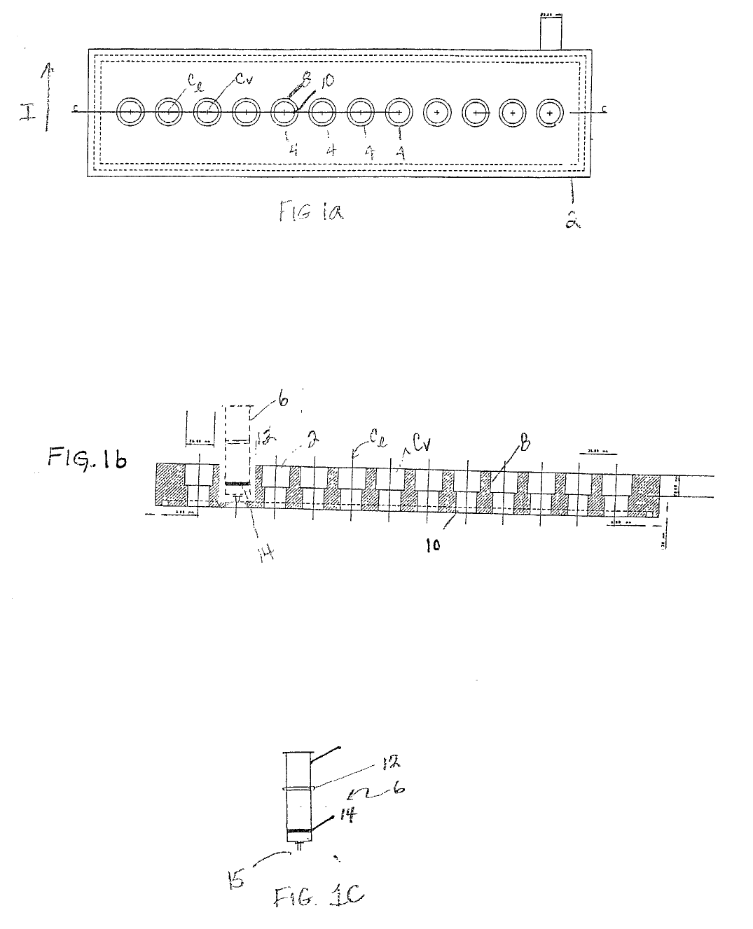

The apparatus for performing the extraction of the method will now be described. Referring first to FIGS. 1a and 1b, a vacuum processing mechanism for isolating nucleic acid comprises a base member 2 having a plurality of multi-diameter chambers 4. Each chamber receives a nucleic acid binding device 6 (FIG. 1c). There are 12 uniformly spaced multi-diameter chambers 4 passing through the base member 2. Each chamber 4 comprises an upper, larger diameter portion 8 connected to a smaller diameter lower portion 10. The portions 8 and 10 of the chambers 4 are concentric.

Each of the binding devices 6 can include an elastomeric band 12 to create and airtight seal within one of the chambers 4 or the band can be placed within each chamber 4. A sorbent 14, which will be described in greater detail herein, is located near the bottom of each of the binding devices 6.

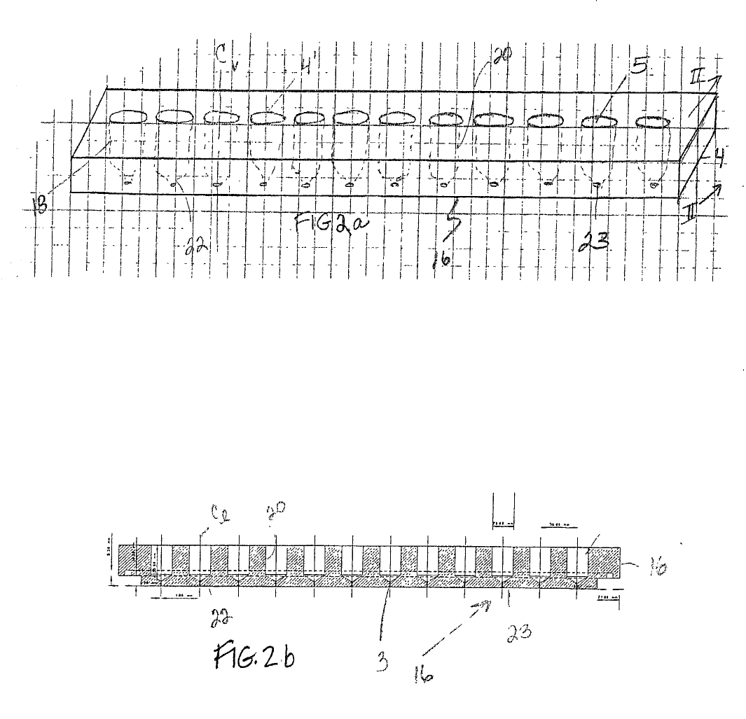

Referring next to FIGS. 2a and 2b, there will be seen an intermediate base 16 containing a plurality of spaced chambers 4′. The vertical center lines C₁ of the chambers 4′ are in alignment across the base 16, parallel with each other, and spaced apart the same distance as the center lines of the chambers 4 in the base member 2. The upper portions of the chambers 4′ are cylindrical, as seen at 20, and the lower portions 22 are conical. The inverted apexes of the conical portions 22 terminate in an orifice 23 of approximately 1 mm in diameter to control the vacuum pressure to each of the chambers 4′. The chambers 4′ of the intermediate base 16, being spaced apart the same distance as the chamber 4 of the base 2 and being of the same size and location, when the upper base 2 is placed on top of the intermediate base 16, the chambers 4 and 4′ are in alignment. That is, when the bottom of upper base is placed upon the top of the intermediate base, the chambers 4 and 4′ are vertically aligned.

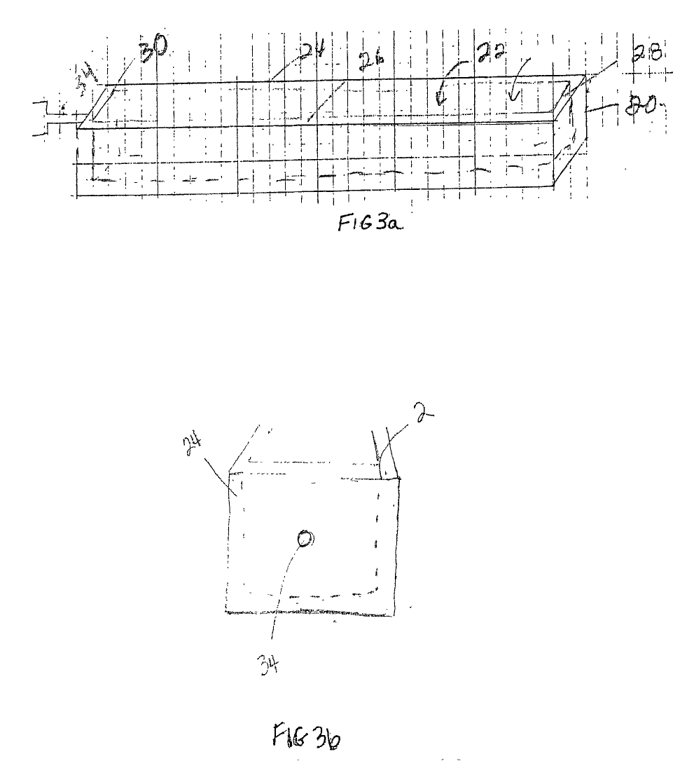

FIG. 3a shows a receiving extraction manifold, generally indicated 20, which has a reservoir 22 formed by walls 24, 26, 28 and 30, respectively. The walls terminate in a flat upper surface 32. In FIG. 3b, which is an end view of the receiving manifold 20, there will be seen an aperture 34 leading from a source of vacuum (not shown). The aperture 34 may also be called a vacuum inlet. The purpose of the reservoir 22 is to receive unbound materials and wash buffers which have filtered through the sorbent 14 in the binding device 6. Although the vacuum inlet 34 is shown at one end of the receiving manifold 20, it may be located any place therein.

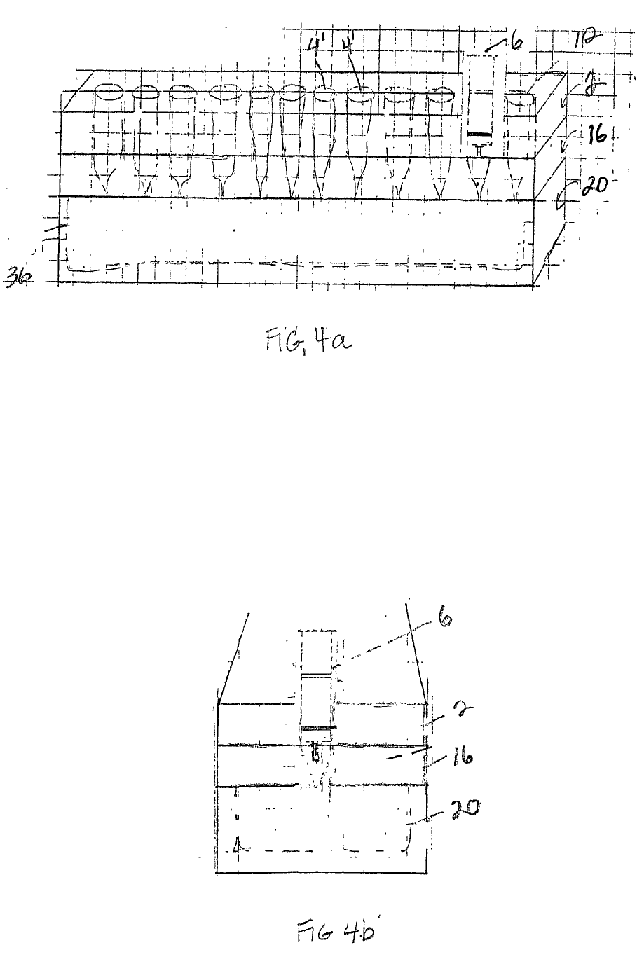

FIG. 4a shows the vacuum processing extraction apparatus assembly, comprising a base 2, the intermediate base 16 and the extraction manifold 20. Vacuum lines and gauges, not shown, are connected to the vacuum inlet 34. This assembly is used for binding and washing steps in nucleic acid purifications. FIG. 4b is an end view of the assembly of the base 2, the intermediate base 16 and the first receiving manifold 20. Grooves, o-rings, or other means, not shown, interlocking means for stacking and securing the assembled elements can be used for making an air tight seal as well as the polished surfaces of each element.

The next set of figures describe the collection apparatus.

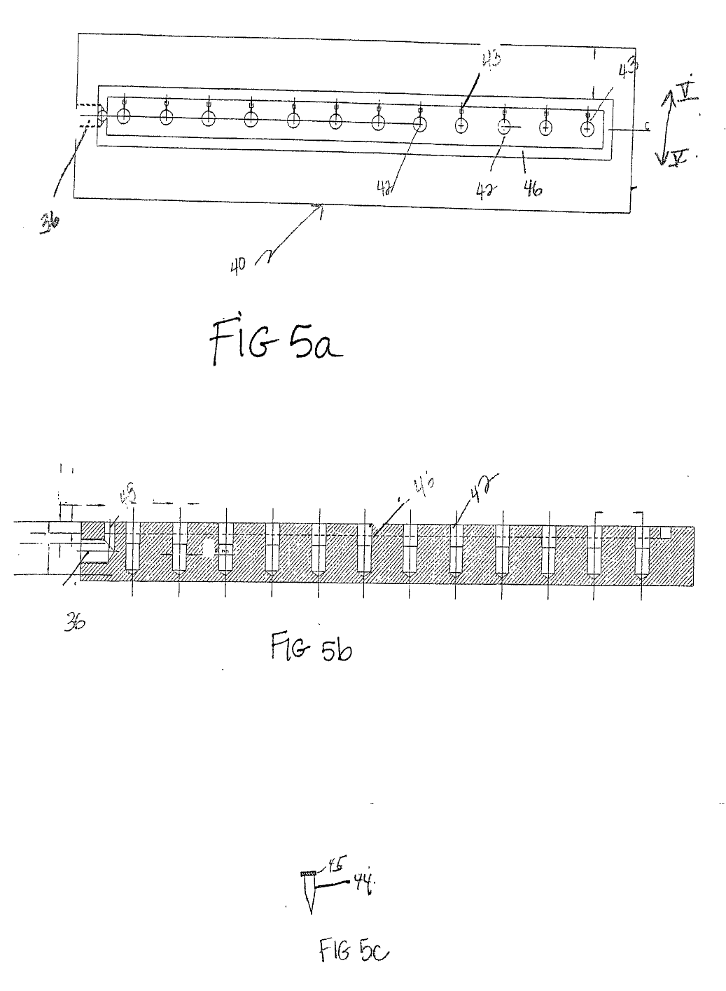

FIG. 5a shows a receiving collection manifold 40, having chambers 42 for holding collection vessels 44. Each chamber 42 has an opening channel 43 to a channel 47 that intersects a vacuum channel 46 connected to the vacuum chamber inlet 36. FIG. 5b shows the collection manifold in cross section.

The collection vessel 44 (FIG. 5c) can be an inverted cone, as is shown 45 (FIG. 4b) and is placed at the bottom of the binding device 6 for collection of the isolated nucleic acids.

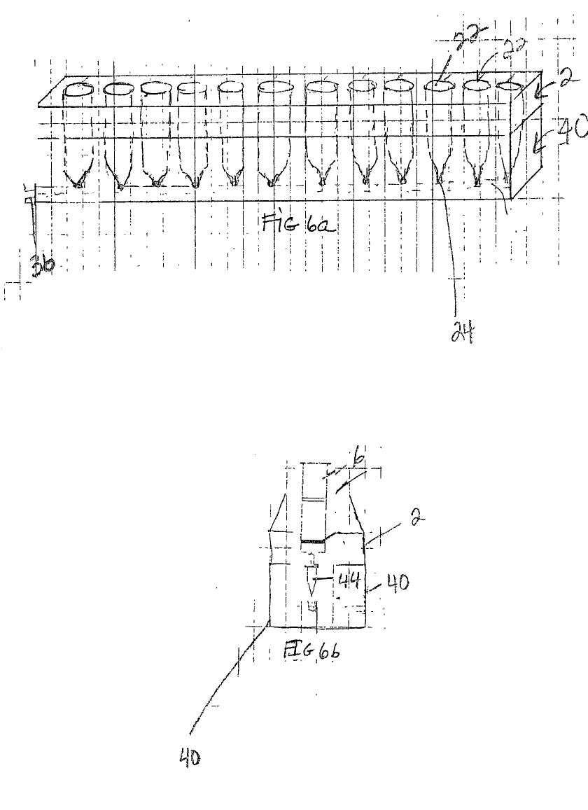

FIGS. 6a and 6b show perspective and end views, respectively, of the collection apparatus, comprising a base member 2 and a receiving collection manifold 40 for the elution of nucleic acids from the nucleic acid binding device 6 directly into collection vessels 44. In this embodiment of the invention, the intermediate base 16 and the extraction manifold 20 are not used.

In operation for the isolation and extraction of nucleic acids, the base member 2 supported on the intermediate base 16, which in turn is seated upon the extraction manifold 20 forming (not shown). The component parts of the vacuum processing mechanism are assembled in this manner for performing the binding and washing steps in the nucleic acid purification process. One nucleic acid binding device 6 is shown schematically in place. The devices are positioned in the chambers 4′ in the chambers in the upper cylinders 8 of the chambers 4 with their lower aperture portions 24 extending downwardly into the lower part 10 of the chambers 4. The receiving collection manifold 40 has a plurality of chambers 42, each of which is adapted to receive a collection vessel 44 in the form of a downwardly pointing, essentially conical receiver with an annulus 46 around its open upper end. Each of the chambers 42 communicates with a channel 46 which connects to a vacuum channel 48, in turn connected to the vacuum inlet 36.

In one embodiment, a vacuum control mechanism 46 and 48 is advantageously incorporated into the collection manifold 40 to control the vacuum pressure to each chamber and minimize the loss of vacuum pressure due to improper connections at each chamber. This vacuum control mechanism is accomplished by utilizing a narrow channel opening from each chamber to a channel that is connected to the vacuum source. The channel from the chamber should be of sufficient diameter to allow vacuum processing to each chamber while avoiding a detrimental pressure drop in any one of the chambers. This vacuum control mechanism is advantageous when processing multiple samples. The control mechanism allows each chamber to utilize the vacuum processing independently of the other chambers. This control is important for when a chamber experiences a detrimental drop in pressure the other chambers will not be seriously effected. In preferred embodiments, the diameter of the channel can be about 1 mm. This channel allows the direct collection of sample into a collection vessel utilizing vacuum processing. The inventive concept of controlling vacuum with a channel from the chamber, as described herein, can be adapted to a varieties of other devices to ensure proper control and use of vacuum processing.

The extraction manifold 20 is connected to the base 2 with the intermediate base 16 between the two to form a vacuum processing assembly, the extraction apparatus, to bind and wash nucleic acids from a sample solution containing nucleic acids. The chambers of the base and intermediate base align. Optionally, a means for providing an air tight seal when vacuum is applied, such as a gasket, can be included between the pieces or the polished surface of the base and the intermediate base can be sufficient to form an air tight seal. Sample is poured into the top or upper funnel of the device, and vacuum is applied via an inlet on the receiving extraction manifold to draw sample through the sorbent or device allowing the nucleic acids to bind to the sorbent under appropriate conditions appropriate for binding. The wash buffers containing filtrate are collected in the receiving extraction manifold. Additionally, if the receiving extraction manifold overfills, a liquid trap placed shortly after the vacuum inlet to restrict any liquid from going into the vacuum system to the vacuum pump. After washing, the receiving extraction manifold and intermediate base are interchanged with the receiving collection manifold containing collection vessels in each chamber. The base and receiving collection manifold comprise the collection apparatus. An elution buffer is applied to the sorbent to disrupt the binding of nucleic acids adsorbed to the sorbent located in the binding device. After a sufficient period of time to allow the nucleic acid binding on the sorbent to be disrupted, vacuum is applied. The elution buffer with nucleic acid sample is collected directly in the collection vessels utilizing vacuum pressure. Centrifugation is not needed for collection.

The apparatus of the present invention reduce the number of manual manipulations other devices utilize, eliminate centrifugation and, therefore, reduce the amount of sample lost. Also, the present invention reduces the amount of attachments and other small pieces that are needed for other vacuum processing methods.

The apparatus can be incorporated into an automatic system with a control means under appropriate vacuum processing conditions. The automation allows for operation of a single or multiple separate nucleic acid purifications with a means of providing the sample to the binding device and a means for delivering the various reagents for washing and elution. The collection apparatus also provides for the elution of nucleic acids directly to collection vessels. The reagents can be delivered from a reservoir to the apparatus by use of pressurized controlled valves. For example, each chamber of the apparatus can be supplied with reagents by a connection to a pump, such as a peristaltic pump, with reagents attached, therefore each chamber can be systematically supplied with washing and elution buffers through a port. The process is controlled by a controlling means and all parts are connected to the controlling means.

Robots can be used to remove the intermediate base and extraction manifold from the base, after the nucleic acid is purified and replace them with the collection manifold aligned with the base for nucleic acid collection. In a preferred mode, the base is removed and placed on top of a collection manifold with vessels located within each chamber and a new base with binding devices located within each chamber is positioned in a sandwich configuration with the intermediate base and receiving extraction manifold to commence the extraction of another set of samples. This minimized the manipulation of elements that are attached to a vacuum source.

The methods of the invention are also amenable to automation for further reduction in manual manipulation. The methods require little human intervention and minimal pipetting. No decanting, centrifugation, precipitation or resuspension of the nucleic acid is required. The methods are also highly efficient, and are thus both cost-effective and suitable for high-throughput screening processes (e.g., genetic screening, drug screening). The method and apparatus also allow for the direct collection into vessels minimizing cross contamination.

The invention is further illustrated by the following non-limiting examples.

### EXEMPLIFICATION

Standard Nucleic Acid Purification Protocol

1. LYSIS. Cells are lysed to liberate or release nucleic acids. Lysis reagents include proteases and chaotropic reagents. A protease solution (Proteinase K) or chaotropic reagent (e.g., guanidinium salt, sodium iodide, potassium iodide), lyses any cell in a test sample to liberate the nucleic acids. Buffers containing chemical lytic agents may also comprise other ingredients which may, for example, create a stable environment for nucleic acids released from cellular or viral materials. Such other ingredients may include salts such as lithium acetate or sodium chloride; or detergents Such as N-lauroylsacrosine or TWEEN. Alternatively, lytic agents may be in the form of mechanical means for lysing cells or virus particles to thereby release nucleic acids. Such means are readily available and may include homogenizers, sonicators or bead beaters.

2. BINDING. Nucleic acids are bound to the sorbent through a physical reversible interaction. The sorbent can be purchased as a devise such as those provided by Qiagen (Valencia, Calif.), Millipore, (Bedford, Mass.) and Macherei Nagel (Düren, Germany). This interaction can be electrostatic resulting from the anionic nature of nucleic acids. Or the interaction can be from other physical interactions such as hydrophobic or affinity. pH and salt concentrations are parameters that can be manipulated to achieve maximum binding.

3.WASHING. Washing is accomplished with appropriate buffers. Washing removes unwanted materials that bind less strongly to the sorbent to be removed.

4. ELUTION. Elution of the nucleic acid from the sorbent in the binding device is accomplished with buffers that disrupt the interaction between the nucleic acid and sorbent. Where ionic interaction predominate, it is usual to elute the nucleic acid in an elution buffer having lower salt concentrations.

### EXAMPLE 2

MODIFICATION OF QIAamp DNA Blood Maxi Kit Protocol From Qiagen, Inc., (Valencia, Calif.) for isolation of genomic DNA from 10 mL (5 mL) of whole blood in a twelve chamber configuration. All reagents are purchased from Qiagen Inc. (Valencia, Calif.).

Notes:

Do not use more than 1×10⁸ cells.

Samples should be equilibrated to room temperature (15-25° C.) before starting.

1. Prepare 12 samples, of 10 mL of blood pipetted into a 50 mL centrifuge tube.

2. Add 500 μl QIAGEN Protease stock solution and then 12 mL of Buffer AL to the sample. Mix thoroughly by vortexing for 30 seconds.

3. Incubate at 70° C. for 10 min.

4. Add 14 mL of ethanol (96-100%) to the sample and mix again by vortexing for 30 seconds.

5. Carefully apply solution onto the QIAamp Maxi column (nucleic acid binding device) located in the chambers of the base, which is attached to the intermediate base and the receiving extraction manifold of the assembly to form a sandwich referred to as the extraction apparatus as is shown in FIG. 4a.

6. Open the vacuum valve and adjust for −0.2 bar. Close the vacuum valve once all the blood is through the sorbent matrix.

7. Add 3 mL of Buffer AW1 to the QIAamp Maxi column. Open the vacuum valve and let the washing buffer through the filter. Repeat with another 3 mL of Aw1 and 2 washes with 3 mL AW2. After all buffer is through the matrix, keep the vacuum valve fully open for 10 minutes to dry the filter completely. The vacuum gauge should read about −0.8 bar. After 10 minutes, close the valve.

8. Remove the intermediate base and the receiving extraction manifold of the assembly and attach the receiving collection manifold containing 2 mL collection vessels in each chamber to the base, with each collection vessel immediately beneath each binding device in the base. (as is shown in FIG. 6a). To each binding device add 1 mL of Buffer AE, wait ten minutes. Apply full vacuum for 30 seconds to retrieve the elution buffer containing the nucleic acid. Repeat with 0.75 mL buffer and then with 0.5 mL buffer. Turn off the vacuum.

DNA yield is measured by absorbency at 260 nm and should fall between 0.1 and 1.0 to be accurate. Samples are diluted to fall within the range of a DNA assay. Purity is determined by calculating the ratio of absorbency at 260 nm to absorbency at 280 nm. Pure DNA has an A260/A280 ratio of 1.7-1.9. Carrier DNA (e.g., poly dA, poly dT and poly dA:dT) can be used when the sample is low-copy (less than 10,000 genome equivalents).

All references described herein are incorporated by reference it their entirety. While this invention has been particularly shown and described with references to preferred embodiments thereof, it will be understood by those skilled in the art that various changes in form and details may be made therein without departing from the scope of the invention encompassed by the appended claims.

What is claimed is:

1. A method of isolating nucleic acids from a biological fluid utilizing vacuum processing and a nucleic acid binding device wherein nucleic acids are collected directly into collection vessels without the use of centrifugation.

2. The method of claim 1, wherein the method of isolating nucleic acids is automated.

3. The method of claim 1, wherein the biological fluid is blood.

4. An apparatus for isolating nucleic acid fluids from a biological fluid, comprising:
a) a base having a plurality of spaced, discreet chambers passing through the base, each chamber having a top opening for receiving a nucleic acid binding device;
b) a first and second manifold which are operationally interchanged during nucleic acid purification, each manifold having an inlet for connection to a vacuum source wherein the receiving vacuum manifold is connected to the base and vacuum is maintained;
wherein the first manifold comprises a reservoir for receiving reagents and is used in the extraction of nucleic acids; and wherein the second receiving manifold having an equal plurality of chamber for receiving collection vessels corresponding in position to the base for collection of nucleic acids into collection vessels and having a channel from the chambers and leading to the vacuum source where in the channel is of sufficient

5. An apparatus of claim 4, wherein the bottom opening of each chamber of the intermediate base is about 1 mm.

6. An apparatus for extracting and isolating nucleic acids from a biological fluid comprising:
a) a first base having a plurality of spaced, discreet chambers passing through the base, each chamber having a top opening for receiving a vial having therein a nucleic acid binding device;
b) an intermediate base having a plurality of chambers corresponding to those in the first base, each chamber in the intermediate base having a top opening and an inverted conical portion terminating in an aperture of sufficient diameter to transmit vacuum to each chamber while avoiding a detrimental pressure drop in a chamber, the intermediate base being of a size and configuration to support the first base on top of it with their respective chambers in vertical alignment; and
c) a receiving extraction manifold having a reservoir communicating with the apertures in the chambers in the intermediate base for receiving reagents used in the extraction of nucleic acids from the vials supported in the base and an inlet in the receiving extraction manifold for connecting the reservoir to a source of vacuum.

7. An apparatus for collecting nucleic acids from a biological fluid comprising:
a) a first base having a plurality of spaced, discreet chambers passing through the base, each chamber having a top opening for receiving a vial having therein a nucleic acid binding device;
b) a receiving collection manifold having a plurality of chambers corresponding in number and location to the chambers in the base, each chamber having a aperture of sufficient diameter to transmit vacuum to each chamber while avoiding a detrimental pressure drop in the chamber, the receiving manifold being of a size and configuration to support the base on top of it their respective chambers in vertical alignment; the chambers the receiving manifold each chamber having a collection vessel; and an inlet in the receiving collection manifold for connecting the chambers to a source of vacuum.

8. An apparatus of claim 6, wherein the bottom opening of each chamber of the intermediate base is about 1 mm.

9. An apparatus of claim 7, wherein the aperature of each chamber of the receiving collection manifold is about 1 mm.

10. A method for purifying nucleic acids using vacuum processing from a biological fluid utilizing the apparatus of claim 4, comprising:
a) incubating the biological fluid with a protease and lysis buffer to liberate nucleic acids contained therein;
b) loading the biological fluid of step (a) into a nucleic acid binding device housed within the chambers of base which is in a sandwich configuration with the intermediate base and first receiving manifold, where the base is on top, the intermediate base is in the middle and the first receiving manifold is on the bottom, wherein the nucleic acids in the solution binds to a sorbent located in the nucleic acid binding device and the liquid filters through the sorbent;
c) washing the sorbent with bound nucleic acid with buffer;
d) collecting the filtrate into the receiving isolation manifold;
e) interchanging the intermediate base and receiving isolation manifold with the receiving collection manifold, containing collection vessels, wherein the base and receiving collection manifold comprise an assembly; and
f) eluting the nucleic acid from the sorbent directly into a collection vessel wherein the method is carried out with vacuum processing without the use of centrifugation.

11. A method for isolating nucleic acids using vacuum processing from a biological fluid utilizing the apparatus of claim 6, comprising:
a) incubating the biological fluid with a protease and lysis buffer to liberate nucleic acids contained therein;
b) loading the biological fluid of step (a) into a nucleic acid binding device housed within the chambers of base which is in a sandwich configuration with the intermediate base and first receiving manifold, where the base is on top, the intermediate base is in the middle and the extraction manifold is on the bottom, wherein the nucleic acids in the solution binds to a sorbent located in the nucleic acid binding device and the liquid filters through the sorbent;
c) washing the sorbent with bound nucleic acid with buffer;
d) collecting the filtrate into the receiving isolation manifold; wherein the method is carried out with vacuum processing without the use of centrifuge.

12. A method for collecting nucleic acids bound to a binding device using vacuum processing utilizing the apparatus of claim 7, comprising the eluting the nucleic acid from the binding device directly into a collection manifold containing collection vessels; wherein the method is carried out with vacuum processing without the use of centrifugation.

13. The method of claim 10 wherein the purification is accomplished by automated vacuum processing.

14. The method of claim 1 wherein the biological fluid is a volume of between about 200 μL and about 10 mL.

15. The method of claim 1 wherein the nucleic acids are DNA, RNA, genomic DNA or viral DNA.

16. The method of claim 10 wherein the biological fluid is whole blood.

17. A method of isolating nucleic acids from a biological sample without the use of centrifugation, comprising removing contaminants from the nucleic acids by vacuum collection while retaining the nucleic acids on a sorbent in a binding device, then releasing purified nucleic acid free of contaminants from the binding device and directly collecting the purified nucleic acids by vacuum processing.
